(19) United States
(12) Patent Application Publication
(10) Pub. No.: US 2013/0096439 A1
(43) Pub. Date: Apr. 18, 2013

(54) METHOD AND SYSTEM FOR CONTACT-FREE HEART RATE MEASUREMENT

(71) Applicants: Kual-Zheng Lee, Chiayi County (TW); Pang-Chan Hung, New Taipei City (TW); Luo-Wei Tsai, Kaohsiung City (TW)

(72) Inventors: Kual-Zheng Lee, Chiayi County (TW); Pang-Chan Hung, New Taipei City (TW); Luo-Wei Tsai, Kaohsiung City (TW)

(73) Assignee: INDUSTRIAL TECHNOLOGY RESEARCH INSTITUTE, Hsin-Chu (TW)

(21) Appl. No.: 13/563,394

(22) Filed: Jul. 31, 2012

(30) Foreign Application Priority Data
Oct. 14, 2011 (TW) 100137384

(51) Int. Cl. A61B 6/00; A61B 5/024

(52) U.S. Cl. 600/479

(57) ABSTRACT

A method and a system for heart rate measurement are provided. The method includes: capturing at least one image, detecting skin-like points by using a skin color detector, labeling the skin-like points and tracking at least one target to be measured, taking statistics on color values of the target at multiple time points, measuring the heart rate through frequency transformation. The method and the system are easy to setup fully an automatic contact-free measurement of multiple persons' heart rates at a time, and applicable in multiple regions of human body for heart rate measurement such as head and neck, arm, and palm regions.

### CROSS-REFERENCE TO RELATED APPLICATION

The present application claims priority to TW patent application Ser. No. 100137384, filed on Oct. 14, 2011.

### BACKGROUND

1. Technical Field

The present disclosure relates to a method and a system for contact-free heart rate measurement, and in particular, to a heart rate measurement technology using ambient light images.

2. Related Art

The heart rate is one of important physiological signals of a human body, so medical professionals or individuals usually measure the heart rate to judge the physiological state. For example, resting heart rate has been identified as an independent risk factor (comparable with smoking, dyslipidemia or hypertension) for cardiovascular diseases.

Heart rate measurement apparatuses in the prior art are mainly contact-based devices, and classified into the following three types.

A First type of contact-based heart rate measurement apparatus is a pulse oximeter, which is based on the red and infrared light absorption characteristics of oxygenated and deoxygenated hemoglobin, in which a light emitter with red and infrared LEDs is used that shines through a reasonably translucent site with good blood flow such as fingers, and then signals are obtained by measuring the light of transmission or reflectance, so as to obtain a blood oxygen concentration and a heart rate value in combination with program computation.

A second type of contact-based heart rate measurement apparatus is a sphygmomanometer, in which a gas bag is inflated to press an artery, so as to block the blood flow, and then the pressure of the gas bag is slowly relieved. In this process, a pressure sensor detects the gas pressure of the gas bag and slight pulses, so as to measure the heart rate and the blood pressure.

A third type of contact-based heart rate measurement apparatus is an electrocardiograph, in which a plurality of adhesive gel patches is pasted on a subject, and the heart rate is detected by electrodes attached to the outer surface of the skin.

Commercial pulse oximeters that attach to the fingertips or earlobes are inconvenient for subjects and the spring-loaded clips can cause pain if worn over a long period of time.

Sphygmomanometers could not measure heart rate at continuous time points. Electrocardiographs are require subjects to wear adhesive gel patches or chest straps that may cause discomfort.

In order to ease the discomfort of subjects and measure multiple persons' heart rates at a time, methods for contact-free heart rate measurement have been developed, which mainly include the following two types.

In the first type of method, a thermal camera is used to sense the information contained in the thermal signal emitted from major superficial vessels of a person and then analyzes the signal to measure the heart rate.

In the second type of method, ambient light images are used to measure the heart rate, in which a camera shoots and detects a human face, and then multiple groups of regions on the human face are labeled manually or a whole face region is used to analyze a periodic variation caused when blood flows through the human face, so as to measure the heart rate.

For the two types of contact-free heart rate measurement methods, the thermal camera is cost expensive. The heart rate measurement using ambient light images can label multiple groups of human faces in one picture in combination with a human face detector and can measure multiple persons' heart rates at a time, the method is merely applicable in front faces and needs to uses an detector with a high computation amount. Furthermore, the human face region includes many meaningless regions without heart rate information, for example, eyebrows, eyes, nares, or beards, which may affect the accuracy.

### SUMMARY

The present disclosure is directed to a method and a system for contact-free heart rate measurement. An embodiment of the present disclosure provides a method for contact-free heart rate measurement, which comprises:

capturing a pattern information;

judging at least one pixel being a skin-like point in the pattern information to output a flag value, and to obtain a color value corresponding to the pixel in the pattern information;

determining the region of at least one target to be measured from the skin-like points to obtain a pixel information of the at least one target;

calculating statistics on targets in a single picture in the pattern information to obtain at least one color value of at least one target region;

obtaining a motion track of at least one target according to a space relation or appearance similarity between the at least one target region at multiple time points;

taking statistics on the pixel information at multiple time points, to transform the pixel information into frequency domain to obtain signal distribution bands and magnitude thereof; and

calculating a heart rate represented by the band according to a time interval between adjacent pictures in the pattern information.

an embodiment of the present disclosure provides a system for contact-free heart rate measurement, which comprises:

a video capture module, configured to capture pattern information comprising videos or images of at least one human skin region of at least one person; and

a heart rate computing module, configured to calculate at least one heart rate.

It is to be understood that both the foregoing general description and the following detailed description are exemplary and explanatory only and are not restrictive of the invention, as claimed.

### BRIEF DESCRIPTION OF THE DRAWINGS

The present disclosure will become more fully understood from the detailed description given herein below for illustration only, and thus are not limitative of the present disclosure, and wherein:

FIG. 1 is a schematic diagram of a system for contact-free heart rate measurement of the present disclosure;

FIG. 2 is a schematic diagram of a first embodiment of a video capture module of the present disclosure;

FIG. 3A is a schematic diagram of a second embodiment of a video capture module of the present disclosure;

FIG. 3B is a schematic diagram of a second embodiment of a video capture module having a reference template of the present disclosure;

FIG. 4A is a schematic diagram of a third embodiment of a video capture module of the present disclosure;

FIG. 4B is a schematic diagram of the third embodiment of a reference template having a reference template of the present disclosure;

FIG. 5 is a schematic diagram of a heart rate computing module of the present disclosure;

FIG. 6 is a schematic flowchart of a method for contact-free heart rate measurement of the present disclosure;

FIG. 7A is a schematic diagram of captured pattern information;

FIG. 7B is a schematic diagram of a region image of pattern information after skin color detection;

FIG. 7C is a schematic diagram of region of labeled targets;

FIG. 8 is a statistical diagram of color trace for color value and frame index;

FIG. 9A to FIG. 9C are statistical diagrams of transformation results of sequence data after frequency transformation;

FIG. 10 is a flowchart of data count adjustment;

FIG. 11 is a schematic diagram of a picture having pattern information of at least three persons; and

FIG. 12 is a schematic diagram in which each person has at least two targets.

### DETAILED DESCRIPTION OF THE DISCLOSURE

Implementation examples are illustrated by the following specific embodiments, and persons of ordinary skill in the art can easily understand the other advantages and efficacies based on the contents disclosed by the specification.

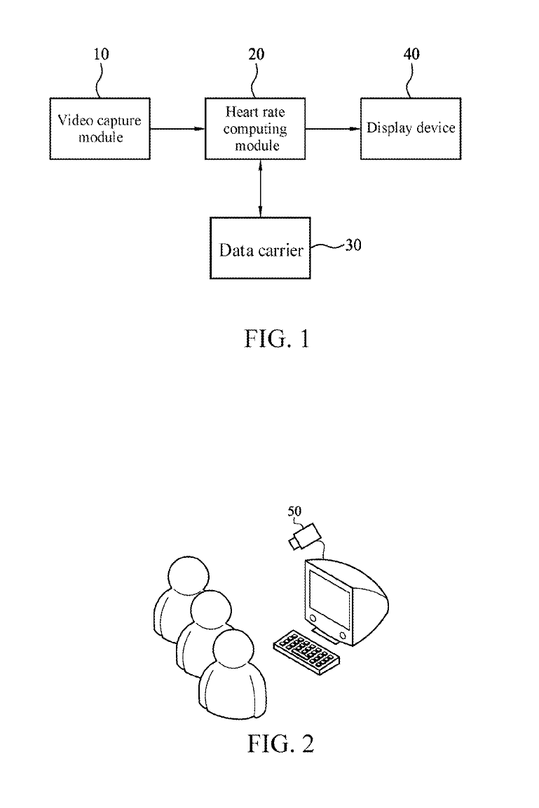

Referring to FIG. 1, a system for contact-free heart rate measurement of the present disclosure includes a video capture module 10, a heart rate computing module 20, a data carrier 30, and a display device 40.

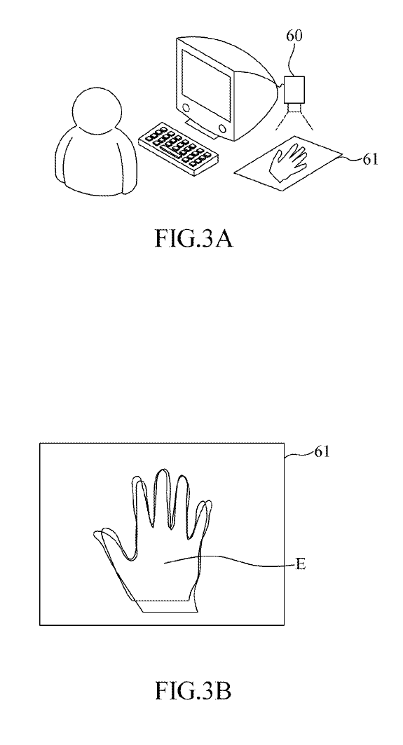

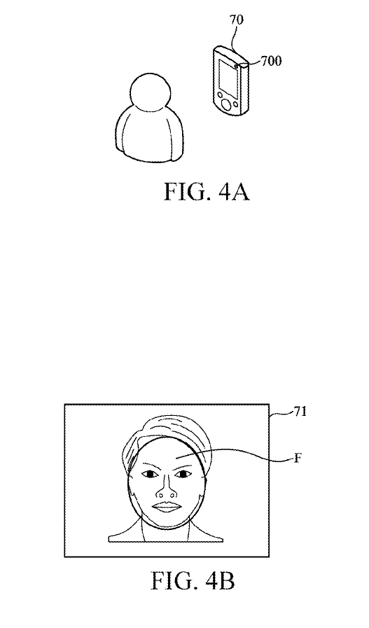

Referring to FIG. 2 to FIG. 4, the video capture module 10 captures pattern information including videos or images of at least one human skin region of at least one person. The pattern information may be in formats such as three primary colors (red, green and blue, RGB for short), True-Color spaces (luminance, chrominance and chroma, YUV for short), or color attribute modes (hue, saturation and value, HSV for short). The video capture module 10 may be a camera 50, a camera 60 having a reference template 61 (as shown in FIG. 3A), a handheld device or tablet PC 70 having a camera 700, or a program that is capable of capturing pictures, video files, or network video streams. The camera 50 and the camera 60 may be network cameras. The handheld device or tablet PC 70 may further have a reference template 71 (as shown in FIG. 4B).

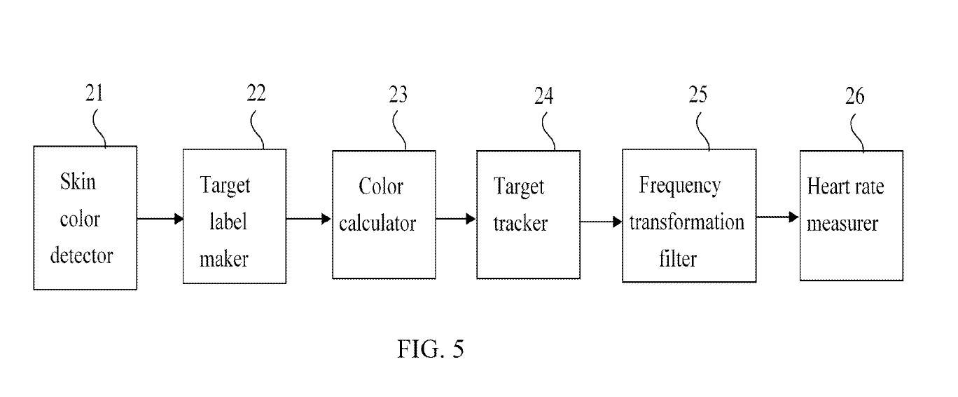

Referring to FIG. 5, the heart rate computing module 20 has a skin color detector 21, a target label maker 22, a color calculator 23, a target tracker 24, a frequency transformation filter 25, and a heart rate measurer 26

The skin color detector 21 is used for judging a pixel that is similar to a human skin color in the pattern information and outputting flag values of skin-like points.

The target label maker 22 is used for obtaining the region of at least one target according to the flag values of skin-like points, and obtaining pixel information of the target.

The color calculator 23 is used for obtaining at least one color value for at least one target region according to the target.

The target tracker 24 is used for tracking the target to obtain a space relation between the target region at multiple time points, so as to obtain a motion track of the at least one target.

The frequency transformation filter 25 is used for taking statistics on data at the multiple time points and transforming the data into frequency domain to obtain signal distribution bands and magnitude thereof. The heart rate measurer 26 is used for calculating a heart rate represented by the band according to a known time interval of adjacent pictures in the pattern information, where the heart rate is a total number of heart beats within a unit time.

The data carrier 30 is used for storing the heart rate or parameters required for computation.

The display device 40 is used for displaying the heart rate.

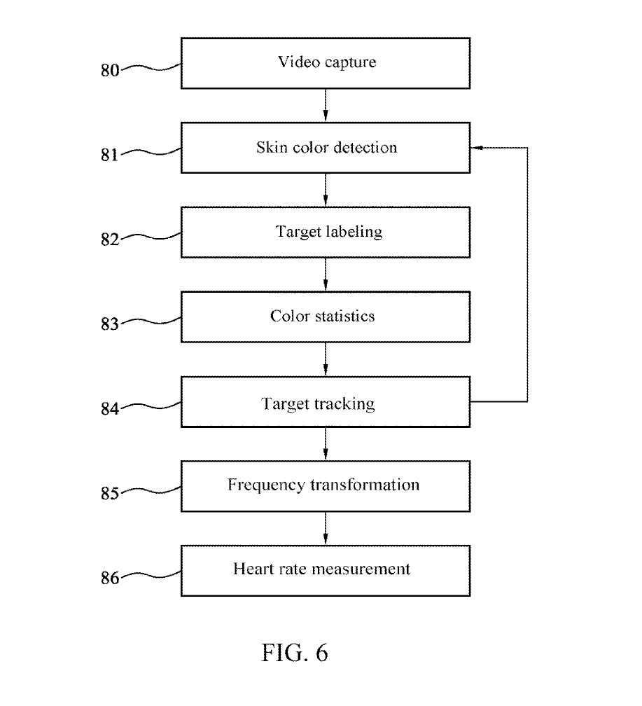

Referring to FIG. 6, a method for contact-free heart rate measurement of the present disclosure includes the following steps.

Video capture 80: The video capture module 10 captures pattern information including videos or images of at least one human skin region of at least one person. The pattern information is obtained from at least one image captured from the frame, at least one opened video file, at least one connected video stream, at least one image shot by a camera, or at least one image shot by a communication device. The pattern information is stored in a time sequence in a readable device to be read for computation. The readable device may be a memory. The format of the pattern information may be red, green and blue (RGB), luminance, chrominance and chroma (YUV), or hue, saturation and value (HSV)

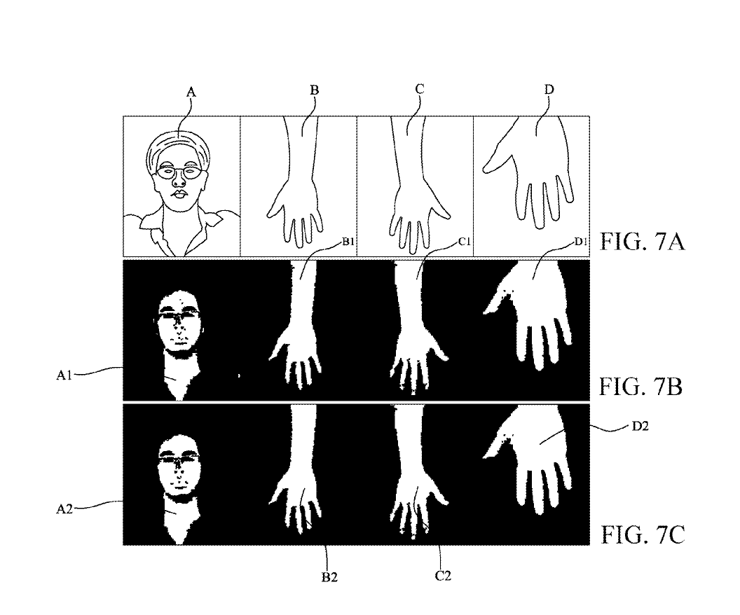

FIG. 7A shows a captured pattern information, and regions of a head and neck A, an inner arm B, an outer arm C, and a center palm D are displayed.

Skin color detection 81: The skin color detector 21 of the heart rate computing module 20 judges a pixel that is similar to a skin color in the pattern information and outputs a flag value whether the pixel in the pattern information is a skin-like point; detects a skin-like point according to the format of the pattern information and according to a skin probability lookup table- trained by neural networks; and obtains all skin-like points in the pattern information and color values corresponding to the skin-like points. The skin probability lookup table is described in detail by K. K. Bhoyar and O. G. Kakde in “Skin color detection model using neural networks and its performance evaluation” (Journal of Computer Science, vol. 6, pp. 955-960, 2010), and details are not described herein.

FIG. 7B shows region images of the pattern information after skin color detection. Referring to FIG. 7B, region images of a head and neck A1, an inner arm B1, an outer arm C1, and a center palm D1 are shown.

Here, t is a time point, Iₜ is set to be video data at the time point t, which is so-called as a single-frame picture or a frame; pₜ={c₁, c₂, . . . , cₖ} is a color of a pixel xₜ on Iₜ. C₁, C₂, . . . cₖ are values of color channels. Taking RGB24 as an example, k=3 and cₖε[0,255]. Color values pₜ of the pixel may be obtained through a video capture process.

Target labeling 82: The target label maker 22 of the heart rate computing module 20 determines the region of at least one target to be measured from the skin-like points and obtains pixel information of the target.

The target labeling 82 may include the following two manners.

According to a first manner, according to the flag values, and through a connected component labeling method, adjacent skin-like points are labeled with the same label to form a region. A region with an excessively large or small area is filtered according to a preset threshold, and a region that falls within the thresholds is regarded as a target. The connected component labeling method is described in detail by L. G. Shapiro and G. C. Stockman in Computer Vision. Upper Saddle River: Prentice Hall, 2001, and details are not described herein.

FIG. 7C shows the region of labeled targets that are obtained through connected components labeling computation, that is, a head and neck A2, an inner arm B2, an outer arm C2, and a center palm D2.

According to a second manner, at least one region of interest is defined. Referring to

FIG. 3 and FIG, 4, the reference template 61 and the reference template 71 are the regions of interest. Referring to FIG. 3A and FIG. 3B, for example, if a palm E is placed at the reference template 61, the camera 60 captures pattern information of the palm E, the pattern information becomes flag values after the step of skin color detection 81, and a skin-like point that is located within the range of the reference template 61 is belonging to a target, and in short, the skin-like points in the overlapped region of the palm E and the reference template 61 is a target. Referring to FIG. 4A and FIG. 4B, the handheld device or tablet PC 70 captures pattern information of a face F, as described above, the skin-like points in the overlapped region of the face F and the reference template 71 is a target. The palm E and the face F are merely used for description, but are not intended to limit the present disclosure. If the obtained pattern information completely covers a reference template, any pattern information can be used.

Color statistics 83: The color calculator 23 of the heart rate computing module 20 takes statistics on targets in one single picture in the pattern information to obtain at least one color value of at least one target region, where the calculation equation is as follows:

$${u}_{t}^{i} = \frac{1}{{n}_{t}^{i}} \sum ( {p}_{t}^{s} \times δ ) , where δ = \{ \begin{matrix}1 & if {x}_{t}^{s} ∈ {R}_{t}^{i} \\ 0 & else ..\end{matrix} ,$$

At a time point t, i is a target region index, uₜⁱ is the obtained color value, Rₜⁱ is a target region obtained by target labeling 82, xₜ^{s} is a skin-like point, pₜ^{s} is a color value corresponding to the skin-like point, and nₜ^{l} is the number of skin-like points of the target region.

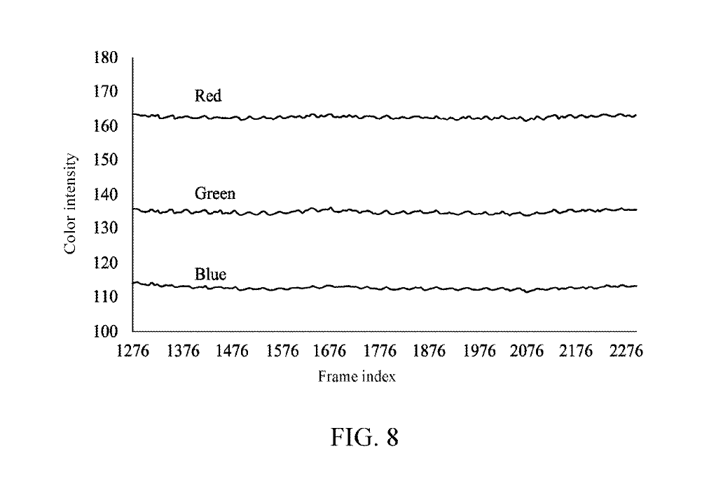

FIG. 8 shows a color statistical result of a head-and-neck region at multiple time points (frames). The region may be an inner arm, an outer arm, or a center palm.

Target tracking 84: The target tracker 24 of the heart rate computing module 20 obtains a motion track of at least one target according to a space relation or appearance similarity between the at least one target region at multiple time points.

For example, regions of targets in each picture of the pattern information are recorded, targets in adjacent pictures and having nearby coordinates are regarded as a single object, and a track of the object is recorded.

Based on the above, at a time point t, a target region of a picture in the pattern information is Rₜ^{l}, the number Mₜ of trackable targets in the picture and target information Oₜ^{j} of the j^{th} tracked target is obtained where j=1,2, . . . Mₜ, in which Oₜ^{j} comprises a set of color values {vₜ^{j}} in each time point and vₜ^{j}=uₜⁱ if the target region Rₜⁱ belongs to Oₜ^{j}.

Frequency transformation 85: The frequency transformation filter 25 of the heart rate computing module 20 takes statistics on data of at least one time point and transforms the data into frequency domain to display signal distribution bands and magnitude thereof. The magnitude is described in detail by B. Boashash in Time-Frequency Signal Analysis and Processing—A Comprehensive Reference (Oxford: Elsevier Science, 2003), and details are not described herein.

Based on the above, the transformation method may be discrete Fourier transformation (DFT), fast Fourier transformation (FFT), discrete cosine transformation (DCT), Hadamard transformation (HT), or discrete wavelet transformation (DWT). The transformation method is described in detail in books of B. Boashash, so the details are not described herein.

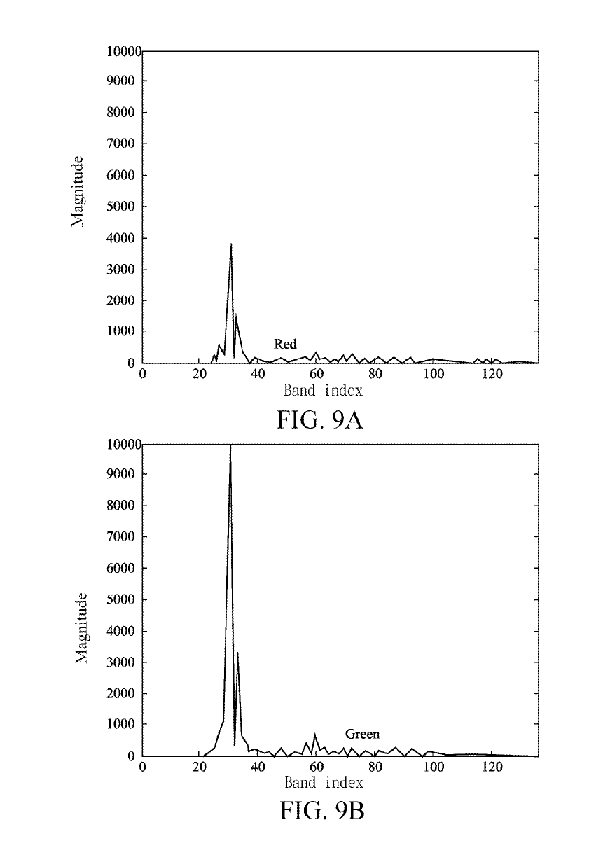

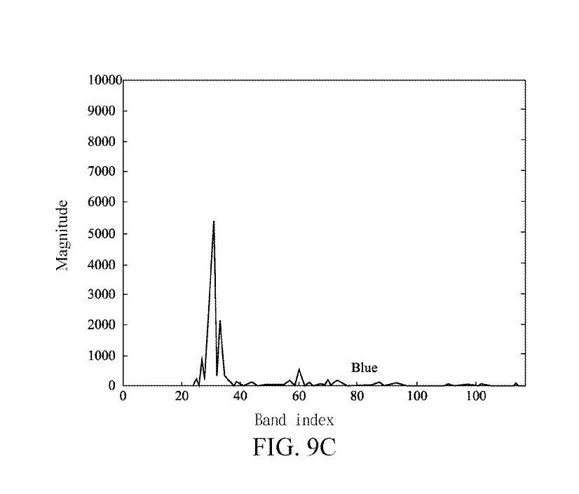

For example, for a j^{th} tracked target, the DFT equation is shown below:

$${X}^{j} ( b ) = {\sum }_{t = 1}^{T - 1} {v}_{t}^{j} {e}^{\frac{- i 2 π tb}{T}} , b = 0 , 1 , … , T - 1 ,$$

where T is the data count to be transformed, t is a time point, e is the base of natural logarithm, i is an imaginary unit, vₜⁱ is the color value of j^{th} target at time point t, X^{j}(b) is magnitude of b^{th} band after the transformation, so a magnitude set corresponding to T−1 bands can be obtained through transformation, that is, forming a power spectrum. Taking RGB as an example, X^{j}(b) includes three groups of magnitude values of color channels R/G/B. As shown in FIG. 9A to FIG. 9C, the horizontal axis is a band index (b), the vertical axis is magnitude, and the transformation method may be discrete Fourier transformation.

In the step of frequency transformation 85, data count T is a main factor that influences the time required for measurement. Therefore, the step further includes a step of data count adjustment to dynamically adjust T, so as to rapidly obtain a frequency transformation result.

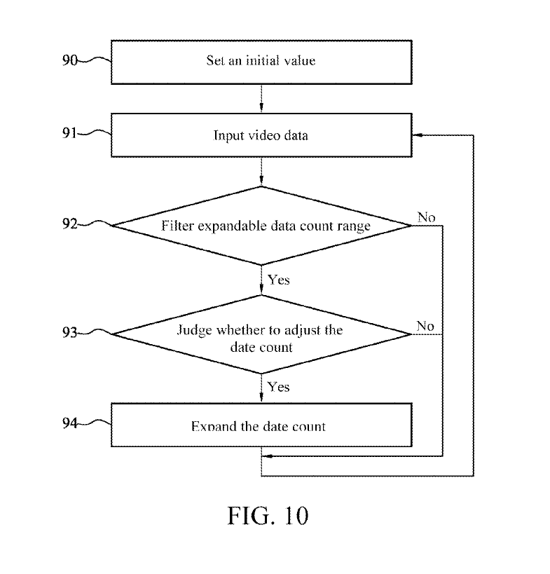

As shown in FIG. 10, the data count adjustment includes the following steps.

Setting an initial value 90: In a preset time period for heart rate measurement, according to a frame rate of a video capture device, a smallest and a largest data counts are obtained, several data counts are selected as preset parameters in ascending order in a time period, a set W={w₁, w₂, . . . ,wₘ} is set to be a pre-selected data count set, where the values are arranged in ascending order and a total number of elements is |W|, and an initial value m=1, so that the data count T=wₘ.

Inputting video data Iₜ 91.

Filtering expandable data count range 92: Judge whether Iₜ meets condition that t≧w₁, m<|W|, and if yes, perform a next step.

Judging whether to adjust the data count 93: Judge whether Iₜ after the foregoing step meets the condition that t≧wₘ₊₁, and if yes, perform a next step.

Expanding the data count 94: Increase the data count as T=wₘ₊₁.

In the steps of filtering expandable data count range 92 and judging whether to adjust the data count 93, if the obtained results are respectively no, return to the step of inputting the video data 91, and the step of expanding the data count 94 may be returned to the step of inputting video data 91, so as to re-start the steps.

In the above steps, in the early period of video capture, a small amount of data is used for the frequency transformation, so the transformation values can be obtained within a very short time. The amount of sampled data is automatically increased with the capture time to improve the accuracy.

Heart rate measurement 86: The heart rate measurer 26 of the heart rate computing module 20 calculates a heart rate H(b) with beat per minute (bpm) unit represented by the band (b) according to a time interval of adjacent pictures in the pattern information. A frame rate of the pattern information is set to be K fps, and the transformation between the band (b) and the heart rate H(b) bpm follows the following:

$$H ( b ) = \frac{60 \times K \times b}{T} .$$

A rational minimum and maximum value for the heart rate are set. For the target Oₜ^{j}, a band bₜ^{j} having the largest magnitude in the rational heart rate range is taken and an equation for transformation is used to calculate a heart rate H(bₜ^{j}) of the target of Oₜ^{j}. Taking a rational heart rate being 40 and 240 as an example, the equation for calculating the band bₜ^{j} is as follows:

$${b}_{t}^{j} = {arg max}_{b} {X}^{j} ( b ) , \frac{40 \times T}{60 \times K} \leq b \leq \frac{240 \times T}{60 \times K} .$$

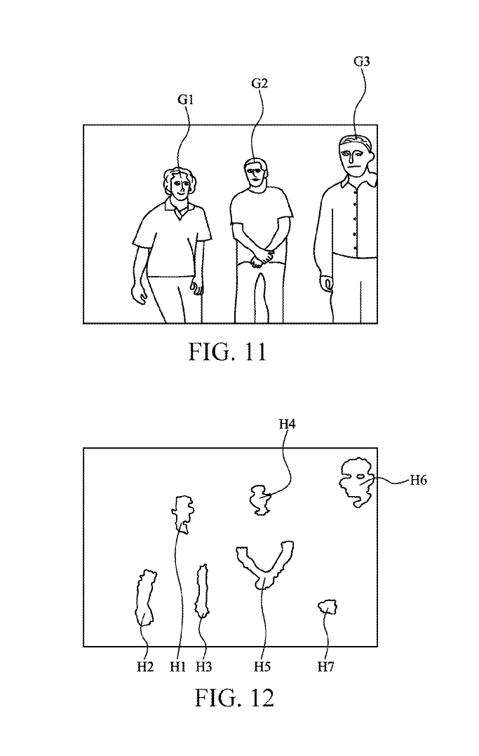

Based on the above, referring to FIG. 11 and FIG. 12, at least three persons G1, G2, and G3 are in the picture of pattern information. Referring to FIG. 12, each person has at least two targets H1, H2, H3, H4, H5, H6, and H7, through the step of target labeling, region ranges for the persons in the picture are obtained, and then it is judged which targets are located in the region of the person. A heart rate can be measured for each target.

Based on the above, in the method and the system for contact-free heart rate measurement of the present disclosure, a video capture module is used to capture an image, and the video capture module may be a camera or an image capture program for a screen picture, a video file, or a network video stream. Through the method and the system, fully automatic contact-free measurement of multiple persons' heart rates at a time can be implemented without using the human face detection algorithm with a high computation amount. Therefore, the method and the system of the present disclosure can be applied in multiple parts of a human body, such as head and neck, arm, and palm regions, to measure the heart rate.

The human face detection algorithm is not required in the present disclosure, so the present disclosure can be applied in multiple parts of a human body, such as head and neck, arm, and palm regions, to measure the heart rate, thereby implementing fully automatic measurement of multiple persons' heart rates.

The present disclosure is applicable in fields such as in general health assessment, ill physiological and mental conditions prediction, polygraph testing, intent identification, smart room, human-computer interaction, and other application fields requiring contact-free heart rate measurement.

The disclosed being thus described, it will be obvious that the same may be varied in many ways. Such variations are not to be regarded as a departure from the spirit and scope of the disclosed, and all such modifications as would be obvious to one skilled in the art are intended to be included within the scope of the following claims.

What is claimed is:

1. A method for contact-free heart rate measurement, comprising:
capturing a pattern information;
judging at least one pixel being a skin-like point in the pattern information to output a flag value, and to obtain a color value corresponding to the pixel in the pattern information;
determining the region of at least one target to be measured from the skin-like points to obtain a pixel information of the at least one target;
calculating statistics on targets in a single picture in the pattern information to obtain at least one color value of at least one target region;
obtaining a motion track of at least one target according to a space relation or appearance similarity between the at least one target region at multiple time points;
taking statistics on the pixel information at multiple time points, to transform the pixel information into frequency domain to obtain signal distribution bands and magnitude thereof; and
calculating a heart rate represented by a band according to a time interval between adjacent pictures in the pattern information.

2. The method for contact-free heart rate measurement according to claim 1, wherein the pattern information has videos or images of at least one human skin region of at least one person, the pattern information is obtained from at least one image captured from a picture, at least one opened video file, at least one connected video stream, at least one image shot by a camera, or at least one image shot by a handheld device or tablet PC; and the pattern information are stored in at least one readable device in a time sequence.

3. The method for contact-free heart rate measurement according to claim 2, wherein the readable device is a memory; and the format of the pattern information is red, green and blue (RGB), luminance, chrominance and chroma (YUV), or hue, saturation and value (HSV).

4. The method for contact-free heart rate measurement according to claim 1, wherein the skin-like points are detected according to a skin probability lookup table trained by neural networks.

5. The method for contact-free heart rate measurement according to claim 1, wherein adjacent skin-like points are labeled with the same label to form a region according to the flag value and a connected component labeling method, a region with an excessively large or small area is filtered according to a preset threshold, and a region that meets the preset threshold is regarded as a target.

6. The method for contact-free heart rate measurement according to claim 1, wherein at least one region of interest is defined, the skin-like points that are located in the region of interest range is a target.

7. The method for contact-free heart rate measurement according to claim 1, wherein the color values of skin-like points are calculated according to the following equation:
$${u}_{t}^{i} = \frac{1}{{n}_{t}^{i}} \sum ( {p}_{t}^{s} \times δ ) , where δ = \{ \begin{matrix}1 & if {x}_{t}^{s} ∈ {R}_{t}^{i} \\ 0 & else . ,\end{matrix} ,$$
wherein, t is a time point, i is a target region index, uₜⁱ is the obtained color value, Rₜⁱ is a target region obtained by target labeling, xₜ^{s} is a skin-like point, pₜ^{s} is a color value corresponding to the skin-like point, and nₜⁱ is the number of skin-like points of the target region.

8. The method for contact-free heart measurement according to claim 1, wherein the region of targets in each picture of the pattern information is recorded, targets in adjacent pictures and having nearby coordinates or similar appearances are regarded as a single object, and a track of the object is recorded.

9. The method for contact-free heart rate measurement according to claim 8, wherein at a time point t, the target region of the picture in the pattern information is Rₜⁱ, the number Mₜ of trackable targets in the picture and target information Oₜ^{j} of the j^{th} tracked target is obtained where j=1,2, . . . Mₜ, in which Oₜ^{j} comprises a set of color values {vₜ^{j}} in each time point and vₜ^{j}=uₜⁱ if the target region Rₜⁱ belongs to Oₜ^{j}.

10. The method for contact-free heart rate measurement according to claim 1, wherein the taking statistics with a transformation method is discrete Fourier transformation (DFT), fast Fourier transformation (FFT), discrete cosine transformation (DCT), Hadamard transformation (HT), or discrete wavelet transformation (DWT).

11. The method for contact-free heart rate measurement according to claim 10, wherein the DFT equation is:
$${X}^{j} ( b ) = {\sum }_{t = 1}^{T - 1} {v}_{t}^{j} {e}^{\frac{- i 2 π t b}{T}} , b = 0 , 1 , … , T - 1 ,$$
wherein T is a data count to be transformed, t is a time point, e is the base of natural logarithm, i is an imaginary unit, vₜ^{j} is the color value of j^{th} target at time point t, X^{j}(b) is magnitude of b^{th} band after transformation, j^{th} is tracked target.

12. The method for contact-free heart rate measurement according to claim 10, wherein further comprises:
Obtaining a smallest and a largest data counts from a fame rate of a video capture device in a preset time period for heart rate measurement, and selecting several data counts as preset parameters in ascending order in a time period, wherein a set W={w₁, w₂, . . . , wₘ} is set to be a pre-selected data count set, wherein the values are arranged in ascending order and a total number of elements is |M|, and an initial value m=1, so that a data count T=wₘ;
inputting a video data Iₜ;
filtering expandable data count range, judging whether Iₜ meets condition that t≧w₁, m<|W|, and if yes, performing a next step;
judging whether to adjust the data count, judging whether Iₜ after the foregoing step meets the condition that t≧wₘ₊₁, and if yes, performing a next step; and
expanding the data count to increase the data count as T=wₘ₊₁.

13. The method for contact-free heart rate measurement according to claim 1, wherein a frame rate of the pattern information is K fps, T is a data count to be transformed, and an equation for transformation between the band b and the heart rate H(b) bpm is as follows:
$$H ( b ) = \frac{60 \times K \times b}{T} .$$

14. The method for contact-free heart rate measurement according to claim 13, wherein a minimum and a maximum values of a rational heart rate being 40 and 240 are set, for the target, a heart rate of the target is calculated through transformation by using a band bₜ^{j} having the largest magnitude in the rational heart rate range in combination with an equation, wherein X^{j}(b) is a magnitude value; and the band bₜ^{j} is calculated according to the following equation:
$${b}_{t}^{j} = {arg max}_{b} {X}^{j} ( b ) , \frac{40 \times T}{60 \times K} \leq b \leq \frac{240 \times T}{60 \times K} .$$

15. A system for contact-free heart rate measurement, comprising:
a video capture module, configured to capture a pattern information comprising videos or images of at least one human skin region of at least one person; and
a heart rate computing module, configured to calculate at least one heart rate according to the pattern information.

16. The system for contact-free heart rate measurement according to claim 15, further comprising: a data carrier, configured to store a heart rate or parameters required for computation; and a display device to display the heart rate.

17. The system for contact-free heart rate measurement according to claim 15, wherein the format of the pattern information is red, green and blue (RGB), luminance, chrominance and chroma (YUV), or hue, saturation and value (HSV).

18. The system for contact-free heart rate measurement according to claim 15, wherein the video capture module is a camera, a handheld device or a tablet PC having a camera, a program capable of capturing a screen picture, a video file, or a network video stream.

19. The system for contact-free heart rate measurement according to claim 18, wherein the camera has a reference template, the camera is a network camera, or the handheld device comprises a reference template.

20. The system for contact-free heart rate measurement according to claim 15, wherein the heart rate computing module comprises: a skin color detector, to judge a pixel that is similar to a human skin color in the pattern information and to output a flag value of a skin-like point; a target label maker, to obtain the region of at least one target according to the flag value of the skin-like point and to obtain a pixel information of the at least one target; a color calculator, to obtain at least one color value of at least one target region according to the at least one target; a target tracker, to track the at least one target to obtain a space relation between the at least one target region at multiple time points, and to obtain a motion track of the at least one target; a frequency transformation filter, to take statistics on data at the multiple time points and transforming the data into frequency domain to obtain signal distribution bands and magnitude thereof; and a heart rate measurer, to calculate a heart rate that is represented by each band according to a known time interval of adjacent pictures in the pattern information.
